(19) United States
(12) Patent Application Publication
(10) Pub. No.: US 2015/0224263 A1
(43) Pub. Date: Aug. 13, 2015

(54) Device For Injecting A Pharmaceutical Liquid, Configured To Be Pre-filled

(71) Applicant: Nemera La Verpillière S.A.S., LA VERPILLIERE (FR)

(72) Inventors: Pascal Dugand, Estrablin (FR); Terry Chatwin, Lathuile (FR); Gaëtan Painchaud, Francheville (FR); Nolwenn Stephan, Lyon (FR); Thierry Rimlinger, L'Isle d'Abeau (FR)

(21) Appl. No.: 14/425,965

(22) PCT Filed: Sep. 4, 2013
(86) PCT No.: PCT/FR2013/052037

§ 371 Date: Mar. 4, 2015

(30) Foreign Application Priority Data
Sep. 4, 2012 (FR) 1258221

(51) Int. Cl. A61M 5/31; A61M 5/00; A61M 5/315

(52) U.S. Cl. CPC A61M 5/3129; A61M 5/315; A61M 5/001; A61M 2205/0238; A61M 2005/3117; A61M 2207/10; A61M 2005/3131

(57) ABSTRACT

An injection device which is configured to be pre-filled, including a vessel for storing the liquid, having two opposing ends, each provided with an opening, referred to as proximal end and distal end, and arranged to receive a sliding piston in order to exert pressure on the liquid. The vessel includes a wall including at least two vertically adjacent layers, each including vertically adjacent plastic material, the first and second layers having different gas permeability.

### FIELD OF THE INVENTION

The present invention relates to the technical field of devices for injecting pharmaceutical liquid, more specifically devices that are prefilled or intended to be prefilled.

### BACKGROUND OF THE INVENTION

Prefilled injection devices such as injection syringes, intended for a single use, are already known. Generally, these syringes are made of glass, due to the fact that glass is known for its properties of impermeability to water and air, and thus allows storage of liquid over a long period, for example over 2 to 3 years. Thus, the medication contained in the syringes does not risk being oxidized by the air or else changing concentration during the storage thereof, which may prove particularly important in the medical field.

Syringes made of plastic are already known, however they are not used very much for producing prefilled syringes, due to the fact that the impermeability to water or to air is not as well known and reliable as in the case of a glass syringe.

It happens that plastic would be a particularly advantageous material for a prefilled syringe, especially for the multiple shape options that plastic offers and also its good breaking strength and impact strength.

For this purpose, one objective of the present invention is in particular to provide an injection device configured to be prefilled while not being made of glass.

### SUMMARY OF THE INVENTION

For this purpose, one subject of the present invention is in particular a device for injecting pharmaceutical liquid, configured to be prefilled, comprising a liquid storage reservoir that has two opposite ends, each provided with an orifice, referred to as proximal end and distal end, and arranged in order to receive a sliding plunger for exerting a pressure on the liquid, the reservoir having a wall that comprises at least two superposed layers, each comprising plastic, referred to as first layer and second layer, having a different permeability to gases.

Thus, it is proposed to produce the reservoir of the injection device using two superposed plastic layers, these two layers being produced so that the superposition thereof may ensure an impermeability to gases that is as good as possible with polymer materials, therefore that is sufficiently impermeable to gases and water in order to produce a prefilled injection device. Specifically, as the two layers have a different permeability to gases, it is possible to choose one layer having a very high impermeability to gases, and another layer having a very high impermeability to liquid, which makes it possible to distinguish the roles by choosing specific and efficient materials for each of the desired functionalities, namely a very efficient plastic for the impermeability to gases, superposed on a very efficient plastic for the impermeability to water. It is therefore proposed to join to a layer having a satisfactory impermeability to liquids, another layer having satisfactory gas impermeability properties, which make it possible to replace a glass device. Thus, according to one example, the first layer, preferably inner layer in contact with the stored pharmaceutical liquid, is highly impermeable to water and may have a relatively low impermeability to gases. In this case, the second layer may be highly impermeable to gases, and have an indifferent impermeability to moisture.

It is understood that the impermeability to gases proposed here makes it possible to prevent the oxidation of the pharmaceutical liquid and the introduction of carbon dioxide capable of changing the pH of the pharmaceutical liquid. It is understood that the impermeability to water includes an impermeability to water in liquid and gaseous form, and thus makes it possible to prevent the evaporation of the water contained in the pharmaceutical liquid capable of modifying the concentration of the medication. It is furthermore understood that the impermeability to water includes an impermeability to an aqueous solution in general.

It is also understood that the layers comprising plastic appear as a replacement for the glass walls conventionally used, and that preferably the device therefore has no glass wall. These layers may each be in multilayer form. Furthermore, the injection device preferably has sufficient impermeability to gases and to water so as to not necessarily need to add additional packaging, such as a blister packaging, in order to ensure impermeability to gases during the storage of the device once prefilled, or systems that absorb gases or water, such as oxygen absorbers, referred to as oxygen scavengers, or water absorbers.

Among the advantages of the use of these layers of plastic instead of glass, it will be noted that the body of the injection device can be easily modified in order to modify the design of the device or in order to add functionalities thereto, unlike cases in which glass is used. Thus, it is possible to reduce the number of parts of complex devices such as autoinjectors. For example, a plastic safety device may easily be incorporated directly onto the injection device, the assembling of the device to a syringe being simpler if both elements are made of plastic than when one of the two is made of glass. The plastic material furthermore makes it possible to facilitate the manufacturing process, the process of cleaning the device before filling (with regard to dust, the presence of tungsten, etc.), ensuring a high chemical inertness of the device in specific environments (for example when the pH is high) or preventing interactions of the material with certain medications, to improve the manufacturing tolerances of the device, therefore the accuracy of the doses injected, to improve the impact strength of the device, to prevent color changes of the device as in the case of glass for example, or else to withstand the gamma sterilization. It is therefore understood that the device described in the present application comprises no conventional glass layer.

It is understood that the first layer may be the inner layer and the second layer positioned on the outer side with respect to the first layer, or vice versa. It is furthermore understood that the layers are superposed and firmly attached to one another, preferably but not necessarily one directly superposed on the other, it being possible for one or more intermediate layers to be between the two.

It will be noted that a different permeability to gases may be understood preferably by, in particular, a different permeability to oxygen and a different permeability to carbon dioxide.

It will furthermore be noted that the injection device presented above differs from a simple vial for storing a pharmaceutical liquid. Indeed, the injection device has two opposite ends, each provided with an orifice. It is intended to receive a sliding plunger on the inside that makes it possible, via the sliding thereof, to inject the pharmaceutical liquid into another container or into the body of a patient. Unlike a vial, such an injection device has no base in which a plastic injection molding point could be made and into which the injection molding of two different materials would be simpler. Indeed, with an injection molding point in the base of the vial, the distribution of the two materials is essentially axisymmetric, therefore relatively controlled, whereas on an injection device provided with two opposite openings that are located along an axis of symmetry, there is no base to ensure this axisymmetric distribution. Thus, the injection device proposed above has two layers of plastic, superposed so that the materials of each layer have a controlled distribution, even though the injection device has no base. Preferably, the first or second layer is in the form of a film, generally produced by an extrusion process. Thus, this film can be overmolded by the other layer or else added to the other layer which has been previously injection molded. Alternatively, the first or second layer is produced by material deposition and/or by surface treatment.

The injection device may additionally comprise one or more of the following features, taken alone or in combination.

The first and second layers are transparent. Thus, the reservoir may be transparent. This is a particular advantage which makes it possible to use plastic that ensures both satisfactory impermeabilities and a transparency of the device. Conventionally, a person skilled in the art was reluctant to use plastic, due to the fact that it often resulted, in order to ensure satisfactory impermeabilities, in non-transparent layers.

The second layer forms a barrier against gases, its oxygen permeability being less than 5 cm³/(m²*day*bar) at 20° C. and at a relative humidity ratio of 65%, preferably less than 1 cm³/(m²*day*bar) at 20° C. and at a relative humidity ratio of 65%. In other words, the second layer allows less than 5 cm³ of oxygen to pass through when it is subjected to a relative humidity ratio of 65%, at a temperature of 20° C., over one day at a pressure of 1 bar, and when it has a surface area of 1m², or even less than 1 cm³ of oxygen. Thus, this property of impermeability to oxygen makes it possible to guarantee a sufficiently long storage of the pharmaceutical liquid in order to use the device as a prefilled device. Preferably, this storage is guaranteed for a duration of greater than two years.

The second layer forms a barrier against gases and comprises a material selected from an ethylene/vinyl alcohol copolymer (EVOH), a polyester or a co-polyester, a polyamide or a co-polyamide, or silicon oxide (SiOx).

The first and/or second layer may be additionally coated with a layer of silicon oxide (SiOx), preferably the layer forming a barrier against gases. Silicon oxide is generally deposited by vapor deposition (referred to as PVD for physical vapor deposition, or CVD for chemical vapor deposition).

The first layer forms a barrier against water, its water permeability being less than 0.2 g/(m²*day*bar) at 23° C. and at a relative humidity ratio of 85%, preferably less than 0.05 g/(m²*day*bar) at 23° C. and at a relative humidity ratio of 85%. In other words, the first layer allows less than 0.2 g (grams) of water, or even less than 0.05 g of water, to pass through when it is subjected to a relative humidity of 85% and to a temperature of 23° C., over one day at a pressure of 1 bar and when it has a surface area of 1 m².

The first layer forms a barrier against water and comprises a cycloolefin polymer. It is understood that the cyclic olefin polymer may be a cyclic olefin copolymer. The expression “cycloolefin polymer” is preferably understood to mean a cyclic olefin polymer (for example COP) or an ethylene/cyclic olefin copolymer (for example COC).

The second layer is on the outer side with respect to the first layer, the first layer forming a barrier against water and the second layer forming a barrier against gases. In other words, the second layer is less permeable to gases than the first layer. These impermeability properties may be obtained either by choosing a particularly suitable material for the layer, or by playing with the thickness of this layer, or by carrying out an appropriate surface treatment if the material is not sufficiently impermeable.

The second layer is on the inner side with respect to the first layer, the first layer forming a barrier against water and the second layer forming a barrier against gases. In other words, the second layer is less permeable to gases than the first layer.

The second layer forms a barrier against gases and comprises at least two plastic-comprising sublayers superposed on one another and firmly attached to one another, one of the sublayers, referred to as the cohesion sublayer, being in contact with the first layer and comprising a material that ensures cohesion with the first layer, preferably by fusion of surface material between this sublayer and the first layer. Preferably, this cohesion sublayer comprises a product compatible with or having a chemical affinity with the first layer, for example owing to a common functional group with the first layer, to a functional group of the same nature as the first layer, to a common product with the first layer or else by being identical to the first layer. For example, this sublayer comprises a cycloolefin polymer in the case where the first layer also comprises a cycloolefin polymer, or else comprises a polypropylene in the case where the first layer comprises a polypropylene.

The second layer additionally comprises, between the two superposed sublayers, a third intermediate tie sublayer, serving as a tie layer (for example by adhesive bonding), for firmly attaching the two other sublayers.

The device comprises an outer layer that forms a marking support, on which graduation information or information relating to the device or to the pharmaceutical liquid may be written, for example by direct printing.

The device comprises an outer layer, or at the very least a layer that covers other inner layers, The device comprises an outer layer, or at the very least a layer that covers other inner layers, the mechanical characteristics of which, such as the tensile modulus, permit an autoclave cycle at 121° C. Thus, this layer makes it possible to maintain the resistance, integrity and geometry of the device during steam sterilization, even if one of the inner layers is liable to melt at 121° C., so much so that the injection device remains intact after cooling. Optionally, a cohesion layer is between this outer layer for the sterilization and another inner layer.

The proximal end orifice is passed through by a plunger rod and the distal end comprises an injection needle. In other words, the injection device is an injection syringe.

The proximal end orifice is passed through by a plunger rod and the distal end comprises a cap, for example a rubber cap, which does not necessarily bear an injection needle. The cap may for example be intended to be replaced by a needle that is added on in the case of a syringe comprising a needle support end fitting (also referred to as a “Luer” fitting) or by a septum in the case of a cartridge. The expression “plunger rod” is understood to mean a rod that is fixedly mounted with respect to the plunger or a part or set of parts having the objective of exerting a force on the plunger in order to discharge the liquid.

The injection device is a single-use injection device. In other words the device is prefilled with a single dose of pharmaceutical liquid and is intended to be thrown away once the dose is injected.

The injection device has a capacity of between 0.5 and 10 ml (milliliters) of pharmaceutical liquid.

The wall of the device comprises a set of layers selected from the following combinations: (COC/EVOH), (COP/EVOH), (COC/polyester), (COP/polyester), (COC/polyamide), (COP/polyamide), (COC/(EVOH/COC)), (COC/(COC/EVOH)), (COP/(EVOH/COP)), (COP/(COP/EVOH)), (COC/(polyester/COC)), (COC/(COC/polyester)), (COP/(polyester/COP)), (COP/(COP/polyester)), (COC/(polyamide/COC)), (COC/(COC/polyamide)), (COP/(polyamide/COP)), (COP/(COP/polyamide)), (COC/(PP/SiOx/polyethylene terephthalate)), (COC/(SiOx/PP/polyethylene terephthalate)), (COC/(SiOx/polyethylene terephthalate/PP)), (COC/(PP/polyethylene terephthalate/SiOx)), (COC/(polyethylene terephthalate/SiOx/PP)), (COC/(polyethylene terephthalate/PP/SiOx)), (COC/(SiOx/COC)), (COC/(COC/SiOx)), (COP/(SiOx/COP)), (COP/(COP/SiOx)), (COC/(SiOx/Polyester)), (COC/(Polyester/SiOx)), (COP/(PP/SiOx/polyethylene terephthalate)), (COP/(SiOx/PP/polyethylene terephthalate)), (COP/(SiOx/polyethylene terephthalate/PP)), (COP/(PP/polyethylene terephthalate/SiOx)), (COP/(polyethylene terephthalate/SiOx/PP)), (COP/(polyethylene terephthalate/PP/SiOx)), (PP/(PP/SiOx/polyethylene terephthalate)), (PP/(SiOx/PP/polyethylene terephthalate)), (PP/(SiOx/polyethylene terephthalate/PP)), (PP/(PP/polyethylene terephthalate/SiOx)), (PP/(polyethylene terephthalate/SiOx/PP)), (PP/(polyethylene terephthalate/PP/SiOx)), (PE/(PP/SiOx/polyethylene terephthalate)), (PE/(SiOx/PP/polyethylene terephthalate)), (PE/(SiOx/polyethylene terephthalate/PP)), (PE/(PP/polyethylene terephthalate/SiOx)), (PE/(polyethylene terephthalate/SiOx/PP)), (PE/(polyethylene terephthalate/PP/SiOx)). Preferably, but not exclusively, the preceding examples are presented in the form: (first layer/second layer). It is understood that it is possible to insert other layers between or around these listed layers, for example a layer of silicon oxide (SiOx). It is also understood that the layers given as an example may include other materials. For example, writing such as (COC/EVOH) denotes an assembly of a layer comprising COC and of a layer comprising EVOH, it being possible for these layers to consist solely of this material or else to additionally include other materials.

Another subject of the invention is a prefilled injection syringe, constituting a device as described above. Another subject could also be a medical cartridge or a medical ampoule. In case of a cartridge (for example for dispensing insulin), the device may be used several times (in this case the septum may be pierced at each new injection by a new needle), on the other hand, once the initial volume has been completely injected, the device is no longer usable.

Another subject of the invention is a process for manufacturing a device for injecting pharmaceutical liquid which is intended to be prefilled, comprising a liquid storage reservoir that has two opposite ends, each provided with an orifice, referred to as proximal end and distal end, and arranged in order to receive a sliding plunger for exerting a pressure on the liquid, comprising the following steps:
a film intended to form a barrier against the gases of the reservoir, referred to as the second layer, is inserted into a mold, and
a material is injected into the same mold in order to form a layer referred to as the first layer, intended to form a barrier against the water in the reservoir.

Another subject of the invention is a process for manufacturing a device for injecting pharmaceutical liquid which is intended to be prefilled, comprising a liquid storage reservoir that has two opposite ends, each provided with an orifice, referred to as proximal end and distal end, and arranged in order to receive a sliding plunger for exerting a pressure on the liquid, comprising the following steps:
a material is injected into a mold in order to form a first layer, intended to form a barrier against the water in the reservoir,
once this first layer is molded, a film intended to form a barrier against the gases of the reservoir, referred to as the second layer, is added around it.

The film forming the second layer may be added by adhesive bonding or heat shrinkage.

It will be noted that the processes proposed above that use a film to form one of the layers are advantageous in that they propose superposing layers without however carrying out bi-injection molding or co-injection molding of material. This aspect is particular advantageous for producing an injection device that has two openings, unlike a vial. Indeed, due to the fact that the injection device has no base, bi-injection molding or co-injection molding of material is problematic since there is no axisymmetric injection molding point in order to distribute the material in a controlled manner. Owing to use of a film for forming one of the layers, the provision of the injection molding point for forming the other layer has little importance and may be elsewhere than in a base. Furthermore, the use of a film may be more advantageous than a surface treatment due to the fact that the film may include a supplementary cohesion layer enabling the film to adhere to any type of surface, whereas the surface treatment may adhere more or less satisfactorily depending on the surfaces.

Optionally, the second layer, or else the first layer, is a multilayer.

### BRIEF DESCRIPTION OF THE DRAWINGS

The invention will be better understood on reading the description that follows, given solely by way of example and with reference to the drawings in which:

FIG. 1 is a longitudinal cross-sectional view of an injection device according to one embodiment;

FIG. 2 is a perspective view of an injection device according to a second embodiment;

FIG. 3 is a perspective view of an injection device according to a third embodiment; and

FIG. 4 is a schematic longitudinal cross-sectional and perspective view of the device from FIG. 3.

### DETAILED DESCRIPTION OF THE INVENTION

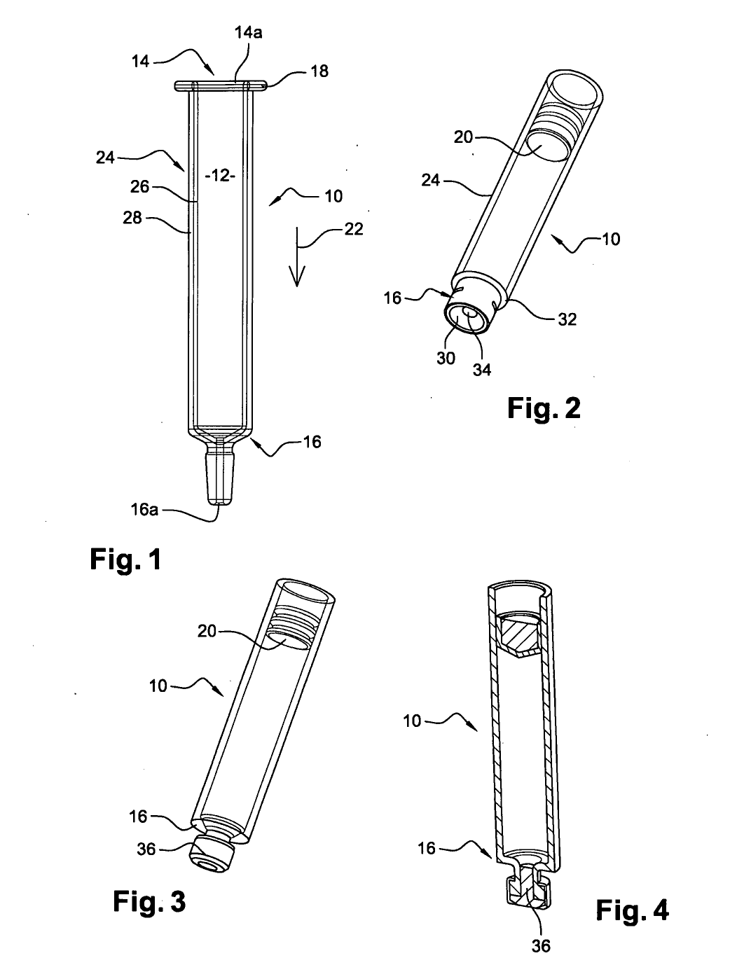

As can be seen in FIG. 1, a device 10 for injecting pharmaceutical liquid may be in the form of a single-use syringe 10. This device 10 is configured to be prefilled, so as to be able to store the pharmaceutical liquid over a relatively long period before injecting it. The device 10 comprises a liquid storage reservoir 12, which has two opposite ends, namely a proximal end 14 and a distal end 16. Each of the ends 14, 16 is provided with an orifice 14a, 16a.

More specifically, the storage reservoir 12 has a tubular shape with circular cross section and has at its proximal end 14, a flange 18, and at its distal end 16, a part intended to receive an added injection needle, introduced into the orifice 16a.

The device 10 is arranged to receive a plunger, similar to the plunger 20 represented in FIG. 2, slidably mounted in the reservoir 12 so as to be able to exert a pressure on the liquid stored in the reservoir 12 in order to make it pass into the injection needle, by sliding of the liquid in the direction indicated by the arrow 22. Generally the plunger 20 is a generally cylindrical axisymmetric part made of rubber or made from another elastically deformable material, mounted in a leaktight manner in the reservoir 12 so that liquid in the reservoir 12 cannot come out through the proximal end 14. The reservoir 12 has inner walls arranged in order to allow sliding cooperation with the plunger, in particular that do not generate excessive friction. In this example, the plunger 20 which is in the reservoir 12 is actuated by a plunger rod which passes through the proximal end 14a, by being pushed by the user's fingers.

The reservoir 12 is defined by a tubular wall 24, comprising a first plastic layer 26 and a second plastic layer 28 that are superposed. The layers 26 and 28 have a different permeability to gases, very particularly to oxygen and to carbon dioxide.

The first layer 26 here forms a barrier against water. Its water permeability is less than 0.2 g/(m²*day*bar) at 23° C. and at a relative humidity ratio of 85%, preferably less than 0.05 g/(m²*day*bar). This first layer 26 comprises a cycloolefin polymer, more specifically a cyclic olefin polymer (for example COP) or an ethylene/cyclic olefin copolymer (for example COC). This first layer 26 is in direct contact with the pharmaceutical liquid stored in the reservoir 12, it prevents the liquid from leaving the reservoir 12 and also prevents water found outside of the injection device 10 from being introduced into the reservoir.

The second layer 28 forms a barrier against gases, its oxygen permeability is less than 5 cm³/(m²*day*bar) at 20° C. and at a relative humidity ratio of 65%, preferably less than 1 cm³/(m²*day*bar) at 20° C. and at a relative humidity ratio of 65%. This second layer 28 comprises a material selected from an ethylene/vinyl alcohol copolymer (EVOH), a polyester or a co-polyester, a polyamide or a co-polyamide, or silicon oxide (SiOx). This second layer 28 is on the outer side with respect to the first layer 26. It is more impermeable to gases than the first layer 26.

The second layer 28 comprises, in this example, two plastic sublayers superposed on one another and firmly attached to one another. The first sublayer, in contact with the first layer 26, is referred to as the cohesion layer, it comprises a material that provides cohesion with the first layer 26, preferably by fusion of material at the surface between this sublayer and the first layer 26. For example, the first layer 26 comprises a cyclic olefin polymer (COP), the cohesion sublayer also comprises a cycloolefin polymer (COP), and the other sublayer comprises a material selected from an ethylene/ethylene vinyl alcohol copolymer (EVOH), a polyester or a co-polyester, a polyamide or a co-polyamide, or silicon oxide (SiOx). Optionally, between the two sublayers forming the second layer 28, an additional sublayer of adhesive may be provided, which is intended to bond the first cohesion sublayer and the other sublayer.

According to one particularly advantageous example, provided on top of the second layer 28 is an outer layer, or at the very least a layer that covers other inner layers, the mechanical characteristics of which, such as the tensile modulus, permit an autoclave cycle at 121° C., which thus makes it possible to ensure the resistance of the injection device 10 during a sterilization.

According to another example, or in combination with this layer, the mechanical characteristics of which permit an autoclave cycle at 121° C., the second layer 28 is covered with an outer layer that forms a marking support, making it possible to identify the syringe and the liquid stored within.

The process for manufacturing the injection device from FIG. 1 will now be described.

According to a first way of proceeding, a film, optionally a multilayer film, intended to form the second layer 28 that forms a barrier against gases is firstly inserted into a mold, then a material is injected into the same mold in order to form the first layer 26.

According to another way of proceeding, a material is injected into a mold in order to form the first layer 26, and once this first layer 26 is molded, a film intended to form the second layer 28 is added around it.

FIG. 2 illustrates an injection device 10 having a reservoir with a composition similar to that from FIG. 1. However, in this example, the device 10 is a medical cartridge, its distal end comprises an added assembly 30, comprising in this example means 32 for attaching the cartridge to another device, such as an autoinjector, and also a central surface area 34 intended to be pierced by a needle during the autoinjection process.

FIGS. 3 and 4 illustrate an injection device 10 similar to that from FIG. 1 and different from a syringe, it is in the form of a medical cartridge having, at its distal end, a septum 36 crimped over the rest of the cartridge. Alternatively, the element 36 could be a removable cap.

It will be noted that the invention is not limited to the embodiments presented above, other embodiments will appear clear to a person skilled in the art. In particular, it is possible to modify the composition of the layers 26 and 28.

1. A device for injecting pharmaceutical liquid, configured to be prefilled, comprising a liquid storage reservoir that has two opposite ends, each provided with an orifice, and referred to as a proximal end and a distal end, and the reservoir being arranged in order to receive a slidable plunger for exerting a pressure on the liquid, characterized in that the reservoir has a wall that comprises at least two superposed layers, each comprising a plastic material, being referred to as a first layer and a second layer, and having a different permeability to gases, the first layer or the second layer being in the form of a film that is overmolded by the other layer.

2. The device according to claim 1, wherein the second layer forms a barrier against gases, its oxygen permeability being less than 5 cm³/(m²*day*bar) at 20° C. and at a relative humidity ratio of 65%.

3. The device according to claim 1, wherein the second layer forms a barrier against gases and comprises a material selected from ethylene/vinyl alcohol copolymer (EVOH), polyester or co-polyester, polyamide or co-polyamide, or silicon oxide (SiOx).

4. The device according to claim 1, wherein the first layer forms a barrier against water and comprises a cycloolefin polymer.

5. The device according to claim 1, wherein the second layer is on the outer side with respect to the first layer, the first layer forming a barrier against water and the second layer forming a barrier against gases.

6. The device according to claim 1, wherein the second layer forms a barrier against gases and comprises at least two plastic sublayers superposed on top of each other and firmly attached to each another, and wherein one of the sublayers, referred to as a cohesion sublayer, is in contact with the first layer and comprises a material that ensures cohesion with the first layer.

7. The device according to claim 1, comprising an outer layer that forms a marking medium.

8. The device according to claim 1, comprising an outer layer, or at least a layer that covers other inner layers, the mechanical characteristics of which permit an autoclave cycle at 121° C.

9. A prefilled injection syringe, constituting a device according to claim 1.

10. A process for manufacturing a device for injecting pharmaceutical liquid, the device being intended to be prefilled and comprising a liquid storage reservoir that has two opposite ends, each provided with an orifice and referred to as a proximal end and a distal end, and the reservoir being arranged in order to receive slidable plunger for exerting a pressure on the liquid, comprising the following steps:
inserting into a mold a film that is intended to form a barrier against the gases of the reservoir, referred to as a second layer,
injecting a material into the same mold in order to form a layer referred to as a first layer, that is intended to form a barrier against the water in the reservoir.

11. The process as according to claim 10, during which the film was produced by an extrusion process, prior to it being introduced into the mold.

12. The device according to claim 2, wherein the oxygen permeability is less than 1 cm³/(m²*day*bar) at 20° C. and at a relative humidity ratio of 65%.

13. The device according to claim 6, wherein the cohesion sublayer comprises a cycloolefin polymer.

14. The device according to claim 6, wherein the material that ensures cohesion is a surface material fused between the cohesion sublayer and the first layer.
